(19) United States
(12) Patent Application Publication
(10) Pub. No.: US 2008/0286778 A1
(43) Pub. Date: Nov. 20, 2008

(54) Method for Investigating Cytosine Methylations in Dna

(76) Inventors: Joern Lewin, Berlin (DE); Juergen Distler, Berlin (DE); Ralf Lesche, Berlin (DE); Matthias Schuster, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 11/885,706

(22) PCT Filed: Mar. 2, 2006
(86) PCT No.: PCT/EP2006/002092

§ 371 Date: May 30, 2008

(30) Foreign Application Priority Data
Mar. 3, 2005 (DE) 10 2005 011 398.2

(51) Int. Cl. C12Q 1/68; C40B 30/04

(52) U.S. Cl. 435/6; 506/9

(57) ABSTRACT

The invention relates to a method for sensitively and specifically detecting cytosine methylations. For this purpose, DNA is first analysed by reacting with the aid of a methylation specific restriction enzyme. In such a way, the background DNA is removed from a reaction preparation. At a next step, a specific conversion of a non-methylated cytosine is carried out, while a methylated cytosine remains unchanged. The converted DNA can be analysed according to different methods, in particular by means of real time PCR method.

### BACKGROUND OF THE INVENTION

The present invention concerns a method to detect 5-methylcytosine in DNA. 5-methylcytosine is the most covalently modified base in the DNA of eukaryotic cells. It plays an important biological role, for example, in transcription regulation, genetic imprinting, and in tumorigenesis (for an overview: Millar et al.: Five not four: History and significance of the fifth base. In: The Epigenome, S. Beck and A. Olek (eds.), Wiley-VCH Verlag Weinheim 2003, P. 3-20). The identification of 5-methylcytosine as an integral part of genetic information is, therefore, of great interest. The detection of methylation is, however, difficult because cytosine and 5-methylcytosine exhibit identical base-pairing behavior. Many of the conventional detection methods, based on hybridization techniques, do not have the capacity to distinguish between cytosine and 5-methylcytosine. Furthermore, the methylation information during a PCR amplification reaction is completely lost.

The conventional methods for methylation analysis basically work according to two significantly different principles. In one case, methylation-specific restriction enzymes are used, and in another, selective chemical conversion is performed of unmethylated cytosine into uracil (so-called: bisulfite-treatment, see also: DE 101 54 317 A1; DE 100 29 915 A1).

Because the use of methylation-specific restriction enzyme requires the recognition of certain sequences, most of the detection methods for methylated DNA are based on bisulfite treatment, which is performed prior to detection or amplification (for example: DE 100 29 915 A1, page 2, lines 35-46). The chemically pre-treated DNA is then in most cases amplified and can be analyzed using different methods (for an overview: WO 02/072880 P 1 ff). Of great interest are methods, which are able to detect methylation sensitively and quantitatively. This is true due to the important role cytosine-methylation has in carcinogenesis, in particular with regard to diagnostic applications. Of special significance are methods that allow for the detection of different methylation patterns in body fluids, such as serum. As opposed to unstable RNA, DNA is often encountered in body fluids. Due to the destructive pathological processes found in cancer, the DNA concentration in blood is, in fact, elevated. A cancer diagnosis through a methylation analysis of body fluids carrying tumor DNA is thereby possible and has been described many times already (see: Palmisano et al.: Predicting lung cancer by detecting aberrant promoter methylation in sputum. Cancer Res. 2000 Nov. 1; 60(21): 5954-8). There exists, however, a problem; in that, in addition to the DNA with methylation patterns typical of a disease-state, one finds a large amount of DNA of the same sequence but with a different methylation pattern. The diagnostic methods must, therefore, have the ability to detect low amounts of specially methylated DNA out of a preponderant background of DNA of the same sequence but different methylation pattern (in the following: background DNA).

The common methods for methylation analyses resolve this problem only insufficiently. Commonly, the chemically pre-treated DNA is amplified using a PCR method. Using either a methylation-specific primer or blocker, a selective amplification of only the methylated DNA (or, for the opposite approach: unmethylated) is warranted. The application of the methylation-specific primer is known as the so-called Methylation-sensitive PCR (“MSP”; Herman et al.: Methylation-specific PCR: a novel PCR assay for methylation status of CpG islands. Proc Natl Acad Sci USA. 1996 Sep. 3; 93(18):9821-6). A method with similar sensitivity is the so-called “Heavy Methyl” method. Thereby, a specific amplification is obtained only of the original methylated (or unmethylated) DNA through the use of methylation-specific blocking oligomers (for an overview: WO 02/072880). The MSP method, as well as, the Heavy Methyl method can be applied as quantifiable real-time variants. They make it possible to detect the methylation status of a few positions directly in the course of the PCR without the necessity for a subsequent analysis of the product (“MethyLight”—WO 00/70090; U.S. Pat. No. 6,331,393). An embodiment of the said real-time methods is the “Taqman” method. This utilizes probes, which carry a pair of fluorescence dye and a quencher. The probes hybridize in a sequence-specific manner to the amplificates and in the course of the subsequent amplification cycles, are degraded through the exonuclease activity of the polymerase. As a result of the separation of the quencher from the dye, a detectable fluorescent signal is produced (see Eads et al.: MethyLight: a high-throughput assay to measure DNA methylation. Nucleic Acids Res. 2000 Apr. 15; 28(8): E32).

A further embodiment of the MethyLight method is the so-called Lightcycler method. In this case, two different probes are deployed that hybridize within the immediate vicinity of each other to the amplificate, and then through the Fluorescence-Resonance-Energy-Transfer (FRET), a detectable signal is produced.

The applicability of these methods for a sensitive and specific detection of methylated DNA from a huge background of unmethylated DNA is, however, limited. There exists a danger that through an unspecific amplification from background DNA false positives could result. False positive signals present, however, one of the most significant problems in the application of methylation technologies for early cancer detection. An increase in the specificity of the methylation detection means, therefore, a meaningful step for the development of the corresponding early detection tests. A reliable, commercial application of the methylation analysis in the area of tumors early diagnostics is thereby facilitated.

In order to increase the specificity of methylation detection, known methods make use of amplification primer or blocking sequences, which contain multiple methylation-specific positions. These sequence requirements allow, however, only the detection of sequences where in a short sequence stretch numerous CpG positions are present. These sequence requirements constrict the applicability of the methods.

On the basis of the given particular biological and medical significance of cytosine methylation and due to the above-mentioned disadvantages of the state of the art, there is a great technical need for the development of powerful methods for sensitive and specific methylation analysis. In the following, a surprisingly simple method is described with which the specificity of methylation detection can be increased.

According to the invention, an enzymatic filter step is conducted before the bisulfite conversion and the subsequent amplification. Through this filter, the background DNA from the reaction mixture is removed. In this filter step, a mixture of different methylation-specific restriction enzymes is used. After the restriction digestion, the bisulfite conversion and amplification/detection are performed in a conventional manner. The enzymatic degradation of the background DNA reduces the danger of false positive results and permits a more specific detection of the methylated cytosine positions.

Indeed the application of the methylation-specific restriction enzymes, as well as, the application of the bisulfite conversion in methylation analyses has been known for a long time (see above). A combination of methylation-specific restriction digestion with a bisulfite conversion and a subsequent amplification has not yet been described. On the basis of the particular biological and medical significance of cytosine methylation and due to the disadvantages of the known methods, the disclosure of this advantageous, new, and surprisingly simple technology presents an important technical advance.

### DESCRIPTION

The method according to the invention for methylation analysis proceeds in the following steps:
1) DNA is isolated from a biological sample,
2) the DNA is subjected to at least with one methylation-specific restriction enzyme, whereby the methylation-specific restriction enzyme degrades the background DNA,
3) the DNA is chemically or enzymatically converted, whereby unmethylated cytosine is converted into thymine or another base, which can be distinguished from cytosine in its base-pairing behavior, whereas methylated cytosine remains unchanged,
4) the converted DNA is analyzed.

In the first step of the method according to the invention, the DNA is isolated from a biological sample. Thereby, the DNA to be examined can be obtained from different sources depending on the diagnostic or scientific question being posed. For diagnostics inquiries, the source material is preferably tissue samples, but also body fluids in particular serum. It is also possible to use DNA from sputum, stool, urine or spinal fluid. DNA can be isolated using standard procedures; isolation from blood can be obtained, for instance, using the Qiagen UltraSens DNA Extraction Kits. In the second step of the method according to the invention, the DNA is converted with the help of at least one methylation-specific restriction enzyme. Through which the methylation-specific restriction enzyme degrades the background DNA. A person skilled in the art knows the numerous restriction enzymes and which can be used according to invention. In particular, the REBASE Database (http://rebase.neb.com/) offers diverse information on methylation-sensitive restriction enzymes. The use of the following enzymes is preferred: HpyCH4 IV, Hha I, Hpa II; HinP1I; Aci I, Zra I, SNAB1, Sal I; Pml1, PaeR7I, Cla I, BspDI, BsaAI, Ava I. The restriction sites of these enzymes are shown in Table 1. All enzymes are commercially available, for example, from New England Biolabs (www.neb.com). The reaction conditions for enzymatic conversion are state of the art and are given, for instance, in the protocols supplied by the manufacturer.

The enzymes have recognition sequences of different lengths. A person skilled in the art knows that through his choice of enzymes, the frequency of fragmentation can be influenced. If he chooses an enzyme, which recognizes a four-base sequence, then clearly, there will be more restriction sites than when using enzymes that have a longer recognition sequence.

In a preferred embodiment, the conversion results from the use of a mixture of various restriction enzymes. So it is warranted that the background DNA is fragmented as completely as possible, and thus, in the subsequent amplification, it is no longer available as a template.

In a preferred embodiment, the enzyme mixture is composed such that all the enzymes being used are active in the buffer and reaction conditions chosen.

In the third step of the method according to the invention, the enzymatically converted DNA is transformed chemically or enzymatically, whereby unmethylated cytosine is converted into thymine or another base, which can be distinguished from cytosine in its base-pairing behavior, whereas methylated cytosine remains unchanged. Preferably, a chemical bisulfite treatment is, thereby, performed. A person skilled in the art knows the differing variations of bisulfite conversion (see for example: Frommer et al.: A genomic sequencing protocol that yields a positive display of 5-methylcytosine residues in individual DNA strands. Proc Natl Acad Sci U S A. 1992 Mar. 1; 89(5):1827-31; Olek, A modified and improved method for bisulphite based cytosine methylation analysis. Nucleic Acids Res. 1996 Dec. 15; 24(24):5064-6; DE 100 29 915; DE 100 29 915). Especially preferred, the bisulfite conversion is performed in the presence of denaturing solvents, such as, Dioxan, and radical scavenger (compare: DE 100 29 915). Further preferred embodiments of bisulfite conversion are described in the German patent applications: DE 103 47 396.3; DE 103 47 397.1; DE 103 47 400.5 und DE 103 47 399.8.

In another preferred embodiment, the DNA is converted not chemically but enzymatically. This is, for instance, conceivable through the application of cytidine deaminases, which can convert the unmethylated cyidtine faster than methylated cytidine. Such an enzyme has been recently identified (Bransteitter et al.: Activation-induced cytidine deaminase deaminates deoxycytidine on single-stranded DNA but requires the action of RNase. Proc Natl Acad Sci USA. 2003 Apr. 1; 100(7):4102-7; compare: German patent application 103 31 107.6).

In the final step of the method according to the invention, the converted DNA is analyzed and thereupon, the methylation status of the original DNA is inferred. The converted DNA can be analyzed by means of the conventional molecular biological methods, for example, with hybridization or sequencing. In a preferred variation, where the methylation status should be detected as sensitively as possible, the converted DNA is first amplified. To this end, a person skilled in the art knows differing variations, for example, ligase chain reactions. In a preferred embodiment, the DNA is amplified, however, using a polymerase reaction. To this end, a variety of embodiments are conceivable, for example, the use of isothermal amplification methods. Especially preferred, however, are polymerase chain reactions (PCR). In an especially preferred embodiment, the PCR is performed using primers, which specifically bind only to sites on the converted sequence that were methylated beforehand (or in a reverse approach: unmethylated). This method is known, for bisulfate-treated DNA, under the name methylation-sensitive PCR (MSP). Thereby, primers are used, which contain at least one 5′-CpG-3′ dinucleotide; preferred are primers, which carry at least three 5′-CpG-3′ sites from which at least one is located at the 3′ end. Therefore, 5′-TG-3′ or 5′-CA-3′ dinucleotides are necessary for the amplification of the unmethylated sequences or the reverse strand (compare: Herman et al.: Methylation-specific PCR: a novel PCR assay for methylation status of CpG islands. Proc Natl Acad Sci USA. 1996 Sep. 3; 93(18):9821-6).

Another especially preferred embodiment for bisulfite-treated DNA is known under the name “Heavy-Methyl” method. Here, a specific amplification only of the methylated (or unmethylated) DNA is accomplished through the application of at least one methylation-specific blocking oligomer. The blocker binds to a 5′-CG-3′ (or a 5′-TG-3′-dinucleotide or a 5′-CA-3′)-dinucleotide and prevents thereby the amplification of the background DNA. The embodiment can be adapted through the choice of polymerase or through the modification of the blocking oligomers so that the degradation or a lengthening of the blocker can be minimized (for an overview: WO 02/072880; Cottrell et al., A real-time PCR assay for DNA-methylation using methylation-specific blockers. Nucleic Acids Res. 2004 Jan. 13; 32(1):e10.).

The detection of the amplificate can be performed using conventional methods, such as, methods measuring the length of sequences like gel electrophoresis, capillary gel electrophoresis, and chromatography (e.g. HPLC). Also mass spectrometry and methods for sequencing like the Sanger method, the Maxam-Gilbert method, and Sequencing by hybridization (SBH) can be used. In a preferred embodiment, the amplificates are detected through primer extension methods (see for example: Gonzalgo & Jones: Rapid quantitation of methylation differences at specific sites using methylation-sensitive single nucleotide primer extension (Ms-SNuPE). Nucleic Acids Res. 1997 Jun. 15; 25(12):2529-31; DE 100 10 282; DE 100 10 280).

In another preferred embodiment, the amplificates are analyzed using hybridization to oligomer arrays (an overview of the array technology can be found in the supplementary issue: Nature Genetics Supplement, Volume 21, January 1999). With such an array, the different oligomers can be arranged on a solid phase in the form of a perpendicular or hexagonal grid. The solid phase surface is preferably composed of silicon, glass, polystyrol, aluminum, steel, iron, copper, nickel, silver or gold.

However, nitrocellulose and synthetic material such as nylon, which can exist in the form of pellets or also resin matrixes, are also possible. The, for example, fluorescently labeled amplificates are hybridized to the bound oligomers, and the unbound fragments are removed. Thereby, it is advantageous, if the oligomer hybridizes to the DNA being analyzed over a stretch of between 12-22 bases, and it should comprise of at least one CG, TG or CA dinucleotide. The fluorescence signal can be scanned and analyzed with software programs (see for example: Adorjan et al., Tumour class prediction and discovery by microarray-based DNA methylation analysis. Nucleic Acids Res. 2002 Mar. 1; 30(5):e21).

Especially preferred is analysis of the amplificates using PCR real time versions (compare: Heid et al.: Real time quantitative PCR. Genome Res. 1996 October; 6(10):986-94, U.S. Pat. No. 6,331,393 “Methyl-Light”). Thereby, the amplification is performed in the presence of a fluorescently labeled reporter oligonucleotide, which hybridizes to a 5′-CG-3′-dinucleotid (or 5′-TG-3′ or 5′-CA-3′-dinucleotid). Thereby, the reporter oligonucleotide binds preferably to the DNA to be examined and indicates the DNA's amplification through an increase or decrease of fluorescence. Thereby, it is especially advantageous when the change in fluorescence is directly used for analysis, and conclusions on the methylation status are drawn from the fluorescence signal. An especially preferred variant is the “Taqman” method. In another especially preferred embodiment, an additional fluorescently labeled oligomer is used that hybridizes proximately to the first reporter oligonucleotide and the hybridization is detected with the use of fluorescence resonance energy transfer (“Lightcycler” method).

Another preferred embodiment of the invention is the simultaneous detection of multiple fragments using the Multiplex PCR to amplify. With this method, one must be certain that not only the primer but also the additional oligonucleotides introduced do not complement each other; thus, the high grade multiplexing is more complex than usual in this case. An advantage of enzymatically treated DNA, however, is that due to the different concentrations of G and C in the two DNA strands a forward primer could never function as a reverse primer; thus, the multiplexing is in turn facilitated, and the above-described disadvantage is approximately counterbalanced. The detection of the amplificates is again possible through different methods. It is conceivable, for instance, to use real time variants. For amplification of more than four genes, detection of the amplificate through other means is recommended. An analysis through arrays is preferred in this case (see above).

Incidentally, it is stressed once again that all known methods for analyzing bisulfite converted DNA can be used also according to the invention. A person skilled in the art can find the specifications on the corresponding methods in scientific publications and in patent literature. A current overview of the possible methods can be found in: Fraga and Esteller: DNA Methylation: A Profile of Methods and Applications. Biotechniques 33:632-649 (September 2002). Particularly special advantages of the method according to the invention arise, as described above, in combination with methods of the sensitive detection of methylation patterns, that is, in particular when using methylation-specific PCR amplification in combination with real time detection methods.

An especially preferred use of the method according to the invention is the diagnosis of cancers or other diseases associated with the alterations of the methylation status. Such diseases are, amongst others, CNS-malfunction, symptoms of aggression or behavioral disorders; clinical, psychological and social consequences from brain impairment; psychotic disorders and personality disorders; Dementia and/or associated syndromes; cardiovascular disease, malfunction and impairment; malfunction, impairment or disease of the gastrointestinal tract; malfunction, impairment or disease of the lung system; damage, inflammation, infection, immune and/or convalescence; malfunction, impairment, or disease of the body as an abnormality in development processes; malfunction, impairment, or disease of the skin, muscle, connective tissue or skeletal tissue; endocrine or metabolic malfunction, impairment or disease; headaches, or sexual dysfunction.

The method according to the invention is, moreover, appropriate for predicting unwanted drug side effects and for distinguishing cell types or tissues or for examining cell differentiation.

The invention is finally also a kit, which consists of at least one methylation-sensitive restriction enzyme and reagents for a bisulfite conversion, as well as, includes optionally also a polymerase, primer, and probes for an amplification and detection.

TABLE 1

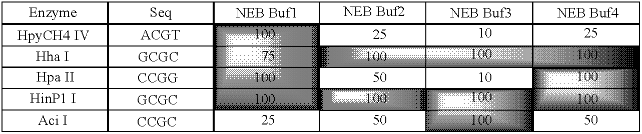

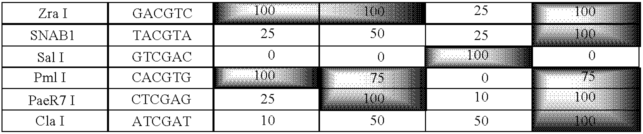

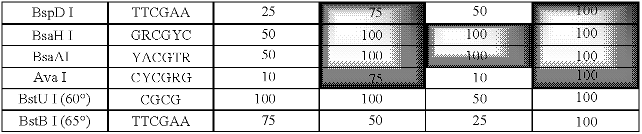

Table 1 shows the restriction sites of different enzymes that can be used for the method according to the invention. The table is taken from New England Biolabs (www.neb.com). The enzymes exhibit their optimum activity in different buffers (NEB Buffer 1-4). The relative activity of the enzymes is given in percentages. The shaded areas indicate the buffer in which the enzymes function most optimally. In a preferred embodiment of the invention, more than one enzyme is used together. Thereby, such enzymes are being preferably combined that exhibit the optimum activity at the same buffer conditions.

### EXAMPLE 1

A diagnostic test is to be developed with which liver diseases, in particular liver cancer, can be detected from a blood sample at an early stage. For that, a DNA sequence is examined, which is methylated only in liver tissue, but exist in other tissues as unmethylated (e.g. muscle, lung, skin, breast) (see below, compare: DE 100 32 529). When the specific methylated sequence is detected in blood, this is an indication that the liver tissue is damaged. A technical problem is, however, that a large amount of unmethylated DNA with the same sequence is present alongside the specifically methylated DNA. In order to develop a specific test, it is, therefore, very advantageous to first specifically degrade the background DNA. For that, as given below, a combination of three restriction enzymes is used. Subsequently, the DNA is subject to a bisulfite conversion, and the converted DNA is amplified in a methylation-specific way. Based on the presence or the amount of the amplificates, conclusions on the presence of the disease can be drawn.

For that, DNA from the blood of an individual first needs to be isolated. For that, different methods are available, for example, Qiagen “UltraSens DNA Extractions-Kits”.

Subsequently, the three restriction enzymes, HpyCH41V (restriction site: ACGT), Aci I (CGCC/GGCG) und HpaII (CCGG), are used according to the manufacturers instructions. Finally, the DNA is bisulfite converted in the presence of denaturing solvents using particular temperature programs and is purified through an ultrafiltration (for details see the German patent applications DE 103 47 396.3; DE 103 47 397.1; DE 103 47 400.5 und DE 103 47 399.8). Afterwards, the converted DNA is amplified using two methylation-specific primers (see below). The amplificates are detected preferably using real time probes (compare (compare: “MethyLight”—WO00/70090; U.S. Pat. No. 6,331,393).

The sequences of the marker and the restriction sites of the enzymes are shown in FIG. 1.

### DESCRIPTION OF THE FIGURES

FIG. 1

FIG. 1 shows the sequence of the liver marker, the restriction sites of the restriction enzymes used, and the binding sites of the primers (binding on the converted sequence). Different primers are shown depending on which of the converted (not anymore complementary) DNA strands is supposed to be amplified. A liver marker was examined, which has already been described in German patent application DE 100 32 529.

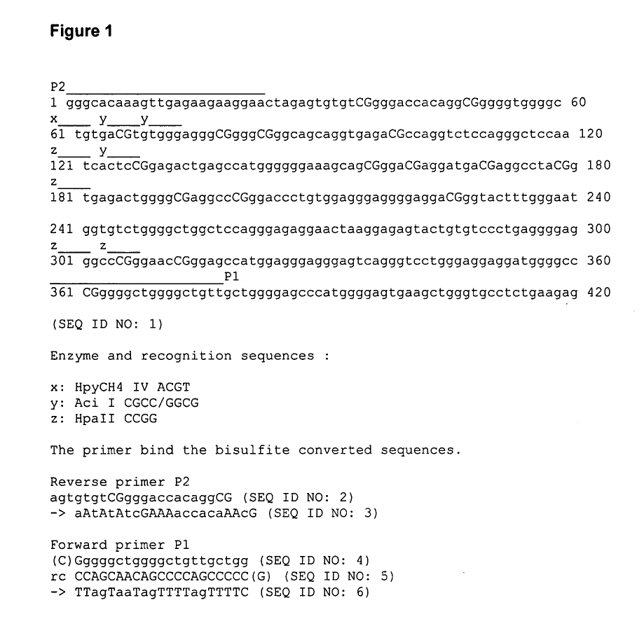

1) Method for methylation analysis characterized by performing the following steps
a) isolating DNA from a biological sample
b) the DNA is subjected to a methylation-specific restriction enzyme, whereby the methylation-specific enzyme degrades the background DNA,
c) the DNA is converted chemically or enzymatically, whereby unmethylated cytosine is converted to thymine or another base, which differs in its base pairing behavior from cytosine while methyl-cytosine remains unchanged,
d) the converted DNA is analyzed.

2) Method according to claim 1 characterized by using one of the following enzymes in the second step: HpyCH4 IV, Hha I, Hpa II; HinP1I; Aci I, Zra I, SNAB1, Sal I; PmI1, PaeR7I, Cla I, BspDI, BsaAI, Ava I.

3) Method according to claim 1 characterized by the use of a mixture of different enzymes in the second step.

4) Method according to claim 3 characterized in that the different enzymes used are active in the same buffer and reaction conditions.

5) Method according to claim 1 characterized by that the conversion is performed in step 3 using a bisulfite.

6) Method according to claim 1, characterized by that the conversion is performed in the third step using a methylation-specific cytosine deaminase.

7) Method according to claim 1, characterized in that the converted DNA is amplified in the fourth step using a polymerase reaction.

8) Method according to claims 7, characterized in that the amplification is performed using a polymerase chain reaction.

9) Method according to claim 7, characterized in that the polymerase chain reaction is performed using methylation-specific primers.

10) Method according to claim 8, characterized in that at least one methylation-specific blocking oligomer is used in the polymerase chain reaction.

11) Method according to claim 7, characterized in that the amplificates are analyzed using methods of length determination, mass spectroscopy or sequencing.

12) Method according to claim 7, characterized in that the amplificates are analyzed using primer extension methods.

13) Method according to claim 7, characterized in that the amplificates are analyzed through hybridization to oligomer arrays.

14) Method according to claim 7, characterized in that the amplificates are analyzed using real time variants.

15) Method according to claim 14, characterized by performing a Taqman or Lightcycler method.

16) Method according to claim 7, characterized in that several fragments are amplified simultaneously using a Multiplex reaction.

17) Use of the methods according to claim 1 for diagnosis of cancers or other diseases associated with an alternation of the methylation status.

18) Use of the methods according to claim 1 for prognosis of unwanted drug side effects, for differentiating of cell types or tissues, or for examination of cell differentiation.

19) A kit consisting of at least one methylation-sensitive restriction enzyme and of reagents for a bisulfite conversion, as well as, including optionally also a polymerase, primer, and probes for an amplification and detection.


SEQUENCE LISTING (6 sequences)

SEQ ID NO: 1 - DNA, 420 bp, Homo Sapiens
gggcacaaagttgagaagaaggaactagagtgtgtcggggaccacaggcgggggtggggctgtgacgtgtgggagggcggggcgggcagcaggtgagacgccaggtctccagggctccaatcactccggagactgagccatggggggaaagcagcgggacgaggatgacgaggcctacggtgagactggggcgaggcccgggaccctgtggagggaggggaggacgggtactttgggaatggtgtctggggctggctccagggagaggaactaaggagagtactgtgtccctgaggggagggcccgggaaccgggagccatggagggagggagtcagggtcctgggaggaggatggggcccgggggctggggctgttgctggggagcccatggggagtgaagctgggtgcctctgaagag

SEQ ID NO: 2 - DNA, 22 bp, Artificial Sequence
  PRIMER
agtgtgtcggggaccacaggcg

SEQ ID NO: 3 - DNA, 22 bp, Artificial Sequence
  PRIMER
aatatatcgaaaaccacaaacg

SEQ ID NO: 4 - DNA, 21 bp, Artificial Sequence
  PRIMER
gggggctggggctgttgctgg

SEQ ID NO: 5 - DNA, 21 bp, Artificial Sequence
  PRIMER
ccagcaacagccccagccccc

SEQ ID NO: 6 - DNA, 21 bp, Artificial Sequence
  PRIMER
ttagtaatagttttagttttc